(19) United States
(12) Patent Application Publication
(10) Pub. No.: US 2003/0232092 A1
(43) Pub. Date: Dec. 18, 2003

(54) Liquid antacid compositions

(76) Inventors: Donald L. Hasenmayer, North Wales, PA (US); John Case, Perkasie, PA (US); Abbie Gentry, Horsham, PA (US); Indu G. Shah, North Wales, PA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/171,707

(22) Filed: Jun. 14, 2002

(51) Int. Cl. A61K 33/12; A61K 33/10; A61K 33/08; A61K 31/045

(52) U.S. Cl. 424/683; 424/688; 424/686; 424/687; 514/738

(57) ABSTRACT

Liquid antacid preparations comprising: a) an active acid neutralizing compound selected from the group consisting of calcium-containing compounds, aluminum-containing compounds, magnesium-containing compounds, and mixtures thereof; and b) an antimicrobial adjuvant selected from the group consisting of propylene glycol in an amount greater than 2 weight percent and less than 15 weight percent, glycerin in an amount from about 15 to about 20 weight percent, and combinations of propylene glycol in an amount from about 3 to about 10 weight percent with glycerin in an amount from about 3 to about 10 weight percent based on the total weight of the preparation are provided.

The present invention relates to liquid antacid preparations comprising at least one acid neutralizing compound and propylene glycol, or glycerin, or a combination of the two. These preparations have an enhanced resistance to microbial contamination.

### BACKGROUND OF THE INVENTION

Antacid preparations are agents that neutralize or remove acid from the gastric contents. Antacid preparations are widely used in the treatment of various gastrointestinal disorders such as peptic ulcers and gastritis. They are also used for the relief of acid indigestion, heartburn, dyspepsia, sour stomach, reflux esophagitis and the like. The clinical use of antacid preparations is based on their ability to neutralize stomach acid and increase the pH of gastric contents.

Antacid preparations used today are made from a variety of active acid neutralizing compounds such as calcium carbonate, sodium bicarbonate, magnesium salts and aluminum salts. Magnesium hydroxide and aluminum hydroxide are the most potent magnesium and aluminum compounds and are often used in combination. In addition, magnesium oxide, magnesium carbonate, aluminum phosphate, magaldrate and magnesium trisilicate are also employed.

Antacid preparations are typically available as liquid suspensions as well as solid dosage forms. In general, suspensions are preferred to tablets or powders since they are more rapidly and effectively solubilized and have a greater ability to react with and neutralize gastric acid.

Liquid antacid preparations are susceptible to microbial contamination, which generally is controlled by adjustment of the pH of the preparation or adding one or more preservatives. However, it is known that preservatives can degrade in solution depending on the pH. One solution to this problem is to add higher amounts of preservatives. However, this adversely effects the taste of the preparation, since preservatives generally have a bitter taste. This, combined with the poor taste of active acid neutralizing compounds, results in lower patient compliance. Accordingly, there is a need for liquid antacid preparations having both acceptable taste and low susceptibility to microbial contamination over the shelf life of the product.

It is known to add propylene glycol or glycerin to acidic based pharmaceutical elixirs and liquids (with pH's on the order of 3.0 to 5.5). When used in such products, the recommended levels of propylene glycol and glycerin are 15 to 30 weight % and greater than 20 weight %, respectively to function as an antimicrobial preservative. Handbook ofPharmaceutical Excipients, 2^{nd} Edition, American Pharmaceutical Association 1994

WO 94/27577 relates to a liquid antacid composition comprising (a) calcium carbonate, (b) short chain alkyl esters of p-hydroxybenzoic acid, (c) benzyl alcohol, (d) optionally but preferably a bis-biguanide compound or a pharmaceutically acceptable salts, esters, isomers, and mixtures thereof, and (e) other excipients. The other excipients may be used to provide an elevated soluble solids content in the composition to enhance preservative efficacy. They may comprise about 60 to about 95 weight % of the composition. Propylene glycol and glycerin are given as examples of the other excipients. However, no amounts of either compound are taught as particularly useful.

Applicants have now discovered that the addition of specific, low levels of propylene glycol or glycerin to liquid antacid preparations provides excellent preservative efficacy.

Applicants have additionally discovered a liquid antacid preparation with excellent preservative efficacy may be formulated substantially free of preservatives using these specific, low levels of propylene glycol or glycerin.

### SUMMARY OF THE INVENTION

The invention provides a liquid antacid preparation comprising: a) an active acid neutralizing compound selected from the group consisting of calcium-containing compounds, aluminum-containing compounds, magnesium-containing compounds, and mixtures thereof; and b) an antimicrobial adjuvant selected from the group consisting of propylene glycol in an amount greater than 2 weight percent and less than 15 weight percent, glycerin in an amount from about 15 to about 20 weight percent, and combinations of propylene glycol in an amount from about 3 to about 10 weight percent with glycerin in an amount from about 3 to about 10 weight percent based on the total weight of the preparation.

### DETAILED DESCRIPTION OF THE INVENTION

Antacids are pharmaceutical products that neutralize at least 5 milliequivalants (mEq) of acid per dose of products. Useful active acid neutralizing compounds include calcium-containing compounds, aluminum-containing compounds, magnesium-containing compounds, and mixtures thereof. Specific examples include calcium carbonate, magnesium carbonate, magnesium trisilicate, aluminum hydroxide, magnesium hydroxide, magnesium oxide, sodium bicarbonate, dihydroxyaluminum sodium carbonatehydrotalcite, and mixtures thereof. Preferred examples include calcium carbonate, magnesium hydroxide, and aluminum hydroxide, and mixtures thereof. Especially preferred are aluminum hydroxide/magnesium hydroxide mixtures. The active acid neutralizing compounds may be utilized as individual powders, e.g. micronized powders, or as amorphous gels. Preferred active acid neutralizing compounds are 13% aluminum hydroxide and magnesium hydroxide 98% in the form of gels.

The total amount of active acid neutralizing compound in the preparation may be, for example, in the range of about 2% to about 70% w/v of the composition. Preferably, the total amount of active acid neutralizing compound in the preparation is in the range of about 14% to about 45% w/v of the composition. When aluminum hydroxide 13%/o/magnesium hydroxide 98% mixtures are employed, the weight ratio of aluminum hydroxide 13% to magnesium hydroxide 98% is preferably in the range of about 10:90 to about 90:10, more preferably 50:50.

An antimicrobial adjuvant, selected from propylene glycol, glycerin, and mixtures thereof, is present in the preparation in a total amount ranging from about 2 to about 20%.

In embodiments where propylene glycol is the sole antimicrobial 3 0 adjuvant, the propylene glycol is present in the preparation in an amount ranging from greater than 2 to less than 15 weight percent of the total weight of the preparation. Preferably, in these embodiments, the preparation comprises about 3 to about 11 weight percent propylene glycol, more preferably about 4 to about 6.25 weight percent propylene glycol. In embodiments where propylene glycol is employed in combination with glycerin, the amount of propylene glycol is preferably from about 3 to about 10%, e.g. from about 4 to about 7%, or in one particular embodiment, the level of propylene glycol is 5%.

Advantageously, these specific, low levels of propylene glycol and glycerin impart excellent preservative efficacy to liquid antacid preparations. This is surprising, in that propylene glycol for example, when present in acidic formulations, is conventionally used at levels of 15 weight % and above to function as an antimicrobial agent. Preservative efficacy of the present preparations is maintained throughout their shelf-life. Preservative efficacy is measured according to <51> Antimicrobial Effectiveness Testing, USP.

Preferably up to about 20 weight percent glycerin is present in the preparation. In embodiments wherein glycerin is the sole anti-microbial adjuvant, the level of glycerin is preferably from about 15 to about 20 weight percent. In embodiments wherein glycerin is employed in combination with propylene glycol, the level of glycerin is preferably from about 3 to about 10%, e.g. from about 4 to about 7%, or in one embodiment the level of glycerin is 4%. Glycerin in particular has been found to impart good taste to the preparation.

A particularly preferred preparation according to the invention employs about 4 to about 6.25 weight percent propylene glycol and about 3 to about 7 weight percent glycerin. This combination has been found to provide excellent preservative efficacy and taste.

The pH of the preparation is preferably in the range of about 7 to about 12, preferably about 7 to about 11, more preferably about 7 to about 9.

The liquid compositions of the invention are aqueous suspensions containing the active ingredients in admixture with pharmaceutically acceptable excipients typically found in aqueous suspensions for oral administration. Such excipients may be suitable suspending agents, for example, sodium alginate, polyvinylpyrrolidone, gum tragacanth, gum acacia, xanthan gum, locust bean gum and cellulose derivatives such as sodium carboxymethylcellulose, microcrystalline cellulose, hydroxy ethylcellulose, methyl cellulose or hydroxypropyl methylcellulose or mixtures thereof Also included may be dispersing or wetting agents such as sorbitan esters or lecithin, antigelling additives, surface modifiers, aqueous or non-aqueous vehicles such as sorbitol solution, ethyl alcohol or fractionated vegetable oils, or diluents.

The preparation may also comprise one or more antimicrobial preservatives. The alkyl esters of para-hydroxybenzoic acid (the parabens, e.g. butylparaben, methylparaben and propylparaben) are preferred and may be used alone or in combination. Generally, the parabens are used in a concentration of about 0.02% w/v. Other antimicrobial preservatives include bis-biguanides and sorbic acid.

In one embodiment the preparation may be substantially free of parabens or bis-biguanides, or other conventional antimicrobial preservatives. This embodiment is advantageous in terms of product taste, as the antimicrobial preservatives, in particular parabens, are known to impart an objectionable taste products. It is therefore desirable to use the lowest level of such preservatives required to impart preservative efficacy to the preparation.

The preparation may also contain flavorings, colorants and/or sweeteners as appropriate. Suitable flavorants include fruit flavors, peppermint, licorice or bubble gum flavors. The sweetening agents may be for example bulk sweeteners or polyols (e.g. maltitol, sorbitol) and/or intense sweeteners such as sucralose, saccharin, aspartame or acesulfame K.

Other active agents may be added to the preparation. For instance, antiflatulents, analgesics, antidiarrheals, H₂ receptor antagonists, proton pump inhibitors, antispasmodic agents may be added as well as other gastrointestinal agents in dosage amounts conventionally used in the treatment of gastrointestinal dysfunction. Examples of suitable gastrointestinal agents include H₂ receptor antagonists, such as famotadine, ranitidine, cimetadine, nizatidine; proton pump inhibitors such as omeprazole or lansoprazole; gastrointestinal cytoprotectives, such as sucralfate and misoprostol; gastrointestinal prokinetics, such as Prucalopride; antibiotics for H. pylori, such as clarithromycin, amoxicillin, tetracycline, and metronidazole; antidiarrheals, such as diphenoxylate and loperamide; glycopyrrolate; antiemetics, such as ondansetron; and analgesics, such as mesalamine. In one embodiment, the additional active agent may be selected from simethicone, bisacodyl, famotadine, ranitidine, cimetidine, prucalopride, diphenoxylate, loperamide, lactase, mesalamine, bismuth, antacids, and pharmaceutically acceptable salts, esters, isomers, and mixtures thereof. In one particularly preferred embodiment, the additional active agent is simethicone.

As used herein, the term “simethicone” refers to the broader class of polydimethylsiloxanes, including but not limited to simethicone and dimethicone. Examples of suitable polydimethylsiloxanes, which include, but are not limited to dimethicone and simethicone, are those disclosed in U.S. Pat. Nos. 4,906,478, 5,275,822, and 6,103,260, the contents of each is expressly incorporated herein by reference.

The liquid antacid preparation may be made using techniques well known in the pharmaceutical industry. For example, the active acid neutralizing compound, antimicrobial adjuvant and other desired excipients and ingredients may be admixed, dispersed in an aqueous vehicle, and homogenized using equipment and procedures known in the art.

The preparation may be administered, for example 1 to 4 times per day. The dosage will depend on the active acid neutralizing compound employed, the condition being treated, and the age and weight of the patient. Typical dosages include about 5-30 mls of the preparation. A suitable dose range for preparations containing aluminum hydroxide 13%/magnesium hydroxide 98% mixtures is from about 170 mg to about 1200 mg per 5 ml, preferably from about 200 to about 700 mg per 5 ml.

The acid neutralizing capacity of the preparations of the present invention is at least about 5 mEq per dose, preferably at least about 10 mEq per dose. For typical formulations of the present invention, the acid neutralizing capacity is at least about 5 mEq per 20 ml, preferably at least about 10 mEq per 20 ml.

In order to further illustrate the present invention and the advantages thereof, the following specific examples are given, it being understood that these examples are intended only to be illustrations without serving as a limitation on the scope of the present invention.

### EXAMPLE 1

Liquid antacid preparations according to the invention were prepared as suspensions containing the following ingredients: aluminum hydroxide USP, magnesium hydroxide USP, propylene glycol USP, propylparaben NF, Glycerine USP, butylparaben NF, sorbitol solution USP 70%, hydroxyethyl cellulose NF, purified water USP, simethicone emulsion USP 30%, sodium saccharin USP, dyes and flavorants. The suspensions were made by first charging and mixing the propylene glycol and/or glycerin, propylparaben and butylparaben, sorbitol solution, hydroxyethyl cellulose, purified water and simethicone emulsion. Then, the aluminum hydroxide was introduced. Next, the magnesium hydroxide was charged. Sodium saccharin was then added, followed by the dyes. Finally, the flavorants were added, and each batch was completed with a final 30 minute mixing step. The finished batches were each passed through a homogenizer at 500 P.S.I.

Samples from each batch were analyzed for preservative efficacy according to USP 24, <51>. The testing was performed promptly on the initial samples from each batch. All samples met the USP Antimicrobial Effectiveness criteria after 28 days of testing. These samples were formulated with paraben levels adjusted to their expected levels at the end of the product expiration period. It can therefor be concluded that the products will meet USP Antimicrobial Effectiveness criteria at the end of their shelf-life (or expiration period).

| | Propylene | | Paraben |
|---|---|---|---|
| | Glycol | Glycerin | Levels (weight |
| Example | (weight %) | (weight %) | % target) |
| 1 | 5.00 | — | 25 |
| 2 | 7.50 | — | 25 |
| 3 | 8.25 | — | 25 |
| 4 | 15.00 | — | 0 |
| 5 | 10.0 | — | 0 |
| 6 | 7.5 | — | 25 |
| 7 | | 20.0 | 0 |
| 8 | | 15.0 | 0 |
| 9 | 10 | 10.0 | 25 |
| 10 | 7.5 | 10.0 | 25 |
| 11 | 5.0 | 10.0 | 25 |
| 12 | 10 | 10.0 | 0 |
| 13 | 5.0 | 10.0 | 25 |
| 14 | 5.0 | — | 25 |
| 15 | 7.50 | — | 25 |
| 16 | 10 | 10.0 | 100 |
| 17 | 5.0 | 10.0 | 25 |
| 18 | 5.0 | 5.0 | 25 |
| 19 | 5.0 | 5.0 | 25 |
| 20 | 4.0 | 6.0 | 25 |
| 21 | 3.0 | 7.0 | 25 |
| 22 | 6.25 | 4.0 | 25 |
| 23 | 5.0 | 4.0 | 25 |
| 24 | 5.0 | 3.0 | 25 |
| 25 | 4.0 | 5.0 | 25 |
| 26 | 4.0 | 4.0 | 25 |

Examples 7 and 8 show embodiments wherein glycerin is employed as the sole antimicrobial adjuvant. These results show that at levels from about 15% to about 20% , glycerin imparts acceptable preservative efficacy to the product without the inclusion of propylene glycol.

The preparations of the invention exhibited excellent preservative efficacy.

### EXAMPLE 2

A liquid antacid preparation according to the invention was compared for taste against commercially available Regular Strength Mylanta® Original Liquid. The preparation according to the invention had the following composition:

| Ingredient | Unit Weight (mg/5 ml) |
|---|---|
| Sorbitol Solution, USP | 953.000 |
| Purified Water USP | 2992.500 |
| Hydroxyethyl Cellulose | 17.000 |
| NF | |
| Simethicone Emulsion, | 70.000 |
| USP | |
| Magnesium Hydroxide, | 204.100 |
| USP | |
| Aluminum Hydroxide, | 787.400 |
| USP | |
| Butylparaben NF | 1.000 |
| Propylparaben NF | 1.500 |
| Glycerin, USP | 230.000 |
| Propylene Glycol, USP | 287.500 |
| Sodium Saccharin, USP | 1.000 |
| N&A FF Antacid #19003 | 20.000 |

What is This??

Using a proto-monadic design, 241 subjects were instructed to swallow a small amount (about 5 ml) of one product. They rated four hedonic attributes, three intensity attributes, and three attributes on “Just Right” scales. They were then instructed to taste the second sample. After tasting the second sample, they were instructed to make an overall preference choice. The two products were distributed in a random, balanced order.

The taste of the preparation of the invention was preferred equally to the Regular Strength Mylanta Original Liquid, 49% to 51%.

We claim:

1. A liquid antacid preparation comprising: a) an active acid neutralizing compound selected from the group consisting of calcium-containing compounds, aluminum-containing compounds, magnesium-containing compounds, and mixtures thereof; and b) an antimicrobial adjuvant selected from the group consisting of propylene glycol in an amount greater than 2 weight percent and less than 15 weight percent, glycerin in an amount from about 15 to about 20 weight percent, and combinations of propylene glycol in an amount from about 3 to about 10 weight percent with glycerin in an amount from about 3 to about 10 weight percent based on the total weight of the preparation.

2. The preparation of claim 1, wherein the active acid-neutralizing compound is selected from the group consisting of calcium carbonate, magnesium carbonate, magnesium trisilicate, aluminum hydroxide, magnesium hydroxide, and mixtures thereof.

3. The preparation of claim 1, wherein the active acid-neutralizing compound is a mixture of aluminum hydroxide and magnesium hydroxide.

4. The preparation of claim 1, wherein the acid neutralizing capacity of the formulation is at least about 5 milli-Equivalents per dose.

5. The preparation of claim 1 wherein the acid neutralizing capacity of the formulation is at least about 5 milli-Equivalents per 20 milliliters.

6. The preparation of claim 1, which is substantially free of parabens.

7. The preparation of claim 1 substantially free of benzyl alcohol.

8. The preparation of claim 1 which is substantially free of bis-biguanides.

9. The preparation of claim 1 having a pH in the range of about 7 to about 12.

10. The preparation of claim 1 in suspension form.

11. The preparation of claim 1 further comprising simethicone.

12. The preparation of claim 1 wherein the antimicrobial adjuvant is propylene glycol, and the propylene glycol comprises about 3 to about 11 weight percent of the preparation.

13. The preparation of claim 1 wherein the antimicrobial adjuvant is a combination of propylene glycol and glycerin, and the propylene glycol comprises about 4 to about 7 weight percent of the preparation.

14. The preparation of claim 1 wherein the antimicrobial adjuvant is a combination of propylene glycol and glycerin, and the glycerin comprises about 4 to about 7 weight percent of the preparation.

15. A liquid antacid preparation comprising: a) an active acid neutralizing compound consisting essentially of a mixture of aluminum hydroxide and magnesium hydroxide; and b) an antimicrobial adjuvant comprising a combination of propylene glycol in an amount from about 3 to about 10 weight percent with glycerin in an amount from about 3 to about 10 weight percent based on the total weight of the preparation.

16. The preparation of claim 15 further comprising simethicone.
